(19) United States
(12) Patent Application Publication
(10) Pub. No.: US 2026/0118653 A1
(43) Pub. Date: Apr. 30, 2026

(54) DOCUMENTATION OF SECTION SURFACES OF HISTOLOGICAL BLOCKS

(71) Applicant: Eberhard Karls Universität Tübingen Medizinische Fakultät, Tübingen (DE)

(72) Inventors: Christian Schürch, Tübingen (DE); Sven Mattern, Tübingen (DE); Robert Asmußen, Tübingen (DE); Ariadne Röhler Bockstedte, Tübingen (DE); Frank Rupp, Tübingen (DE)

(21) Appl. No.: 19/426,617

(22) Filed: Dec. 19, 2025

Related U.S. Application Data
(63) Continuation of application No. PCT/EP2024/065934, filed on Jun. 10, 2024.

(30) Foreign Application Priority Data
Jun. 21, 2023 (DE) 10 2023 116228.4

(51) Int. Cl. G02B 21/36; G16H 30/20; H04N 23/61; H04N 23/66

(52) U.S. Cl. CPC G02B 21/365; G16H 30/20; H04N 23/61; H04N 23/66

(57) ABSTRACT

The invention relates to a device for automated capturing of at least one image of a block which comprises a sample embedded for histological purposes and an identification code. The device comprises a holder for the block, a camera unit comprising at least one camera, and a control unit. The camera unit is configured to repeatedly capture an image from the region of the holder and to send it to the control unit. The control unit is configured to receive and analyze each image. The control unit, provided that the identification code has been recognized, instructs the camera unit to capture at least one image of the sample embedded in the block. Further, the invention relates to a method of automated capturing at least one image of a block which comprises a sample embedded for histological purposes and an identification code, wherein the method comprises capturing an image from the region of the holder, analyzing the image from the region of the holder, repeating the preceding steps until the identification code is recognized, and capturing at least one image of the sample embedded in the block.

This application is a continuation of international patent application PCT/EP2024/065934, filed on 10 Jun. 2024 designating the U.S., which international patent application has been published in German language and claims priority from German patent application 10 2023 116 228.4, filed on 21 Jun. 2023. The entire contents of these priority applications are incorporated herein by reference.

### TECHNICAL FIELD

The invention relates to the field of automated digital imaging, in particular to the field of documentation of section surfaces of histological blocks by means of such imaging. The invention particularly relates to a device and a method for automated capturing of at least one image of a block which comprises a sample embedded for histological purposes and an identification code, as well as to a computer program product by which the device can be caused to carry out the method.

### BACKGROUND

Histology is a method used in science and medicine in various ways for the microscopic examination of samples. In this method, a sample is embedded in a block made of embedding material, which usually is a tissue sample of a living being or may represent a sample of any material.

As embedding material, for example, paraffin or plastic polymers can be used. The embedding material is poured in liquid form around the sample and then hardened. It stabilizes the sample for the preparation of sections. The casting of the block takes place in a so-called embedding cassette, which gives the block its shape and in which the block may remain after hardening. The embedding cassette can be labeled with information relating to the sample, in particular with information regarding its origin. Such information can also be contained in a barcode or identification code which is attached to the embedding cassette or forms part of the embedding cassette.

By means of a microtome, sections are prepared by the user, for example with a thickness of about 0.5 to 10 μm, and placed on an object slide. After further processing, which may include staining of certain tissue regions, the section of the sample can be viewed under a microscope.

During or after the cutting process, it may occur that parts of the sample are lost from the section, such that they are not visible during final observation of the section under a microscope. It may also occur that a section is assigned, for example, to the wrong patient or to the wrong block. Conclusions drawn from the sample in the section would then be attributed to the wrong block or the wrong patient. These are basically problems in science and medicine, particularly in pathology.

To counteract this, the section surface of the block can be optically compared with the section located on the object slide in order to determine whether the section is complete. Such comparison is often only possible on the basis of enlarged images of the sample, since small losses in the section are difficult or impossible to recognize with the naked eye. A comparison can be carried out manually or by producing images of the section surface.

A manual comparison of the sections with the corresponding block is very time-consuming and labor-intensive, since the block must first be obtained, for example from an archive. Moreover, such a procedure requires the presence of the user at his or her workplace in order to have physical access to the blocks.

U.S. Pat. No. 11,257,216 B2 discloses a device for capturing images of blocks and object slides, wherein first the section surface and the object slide are captured and then, if applicable, an existing barcode. By means of the barcode, the images are then digitally archived and assigned to a patient. The captured images can subsequently be compared automatically. However, the capturing of images of the section surface of the block is a time-consuming process, especially since, as a rule, a large number of blocks occurs and a block may have to be captured several times.

It is an object of the present invention to eliminate the disadvantages of the prior art mentioned above by providing a device which enables a high block throughput with minimal time and labor effort for the user.

### SUMMARY

According to the invention, the object is achieved by providing a device for automated capturing of at least one image of a block which comprises a sample embedded for histological purposes and an identification code, wherein the device comprises:
a holder for the block,
a camera unit comprising at least one camera,
a control unit,
   wherein the camera unit is configured to repeatedly capture an image from the region of the holder and to send it to the control unit,
   wherein the control unit is configured to receive and analyze each image,
   wherein the control unit, provided that the identification code has been recognized, instructs the camera unit to capture at least one image of the sample embedded in the block.

According to the invention, the sample may be any kind of material the microscopic examination of which is of interest and which is in a cuttable state or has been brought into a cuttable state. Preferably, the sample is tissue of a living being.

In the present context, a block or a histological block is to be understood as at least the sample embedded in an embedding material for histological purposes. Embedding materials may be paraffin or plastic polymers. The shape of the block is not limited to a specific form. The block may, for example, be cuboid, cubic, disk-shaped, or spherical and is preferably cuboid or cubic. However, the shape may also be asymmetrical or irregular.

The block may further include an embedding cassette in which the embedding material is anchored. The embedding cassette may be a commercially available embedding cassette for histological purposes. Such an embedding cassette may have dimensions of about 42 mm×29 mm×5 mm. The surface of the block on which sections have been made forms the section surface. At the section surface, the embedded sample is visible.

According to the invention, an identification code is to be understood as any kind of optically readable information by means of which it is possible to identify the block. The information may be present, for example, in the form of a sequence of letters or numbers, a barcode, a QR code, a DataMatrix code, a color code, a color marking, or a similar format. The identification code may be positioned at any arbitrary location of the block, with the exception of a location at which the identification code covers-up the embedded sample or a part thereof.

According to the invention, a holder for the block is to be understood as any structure that can be used as a mount or support for a block. The holder may clamp or snap the block into place or merely serve as a position marking on which the block must be placed so that the camera unit can capture an image of it.

According to the invention, the camera unit comprises at least one camera. Within the context of the present invention, a camera is also to be understood as including a scanner. The camera is positioned and the optical system of the camera is configured in such a way that the camera can capture a sufficiently resolved image from the region of the holder. The image is sufficiently resolved when the resolution enables both the control unit to recognize an existing identification code and a healthy human eye to recognize a loss of sample material when comparing the section surface of the block with the corresponding section on an object slide. Loss of sample material from the section may occur, for example, due to falling out or tearing out of individual areas during cutting or handling of the section. The camera unit may be of decentralized configuration, i.e., it may comprise several components that are spatially and/or technically separated from one another.

The control unit is a technical device, preferably a computer, which comprises at least a temporary memory, for example a so-called working memory, and may further comprise a permanent memory. In addition, the control unit comprises a processor (CPU). The control unit may be of decentralized configuration, i.e., it may comprise two or more secondary control units each having their own CPU. The secondary control units may be spatially and/or technically separated from one another and may perform different functions of the control unit or, in other words, control different components of the device.

The term “capture” is to be understood here as the optical acquisition or taking of an image, its translation into a binary code, and its temporary or permanent storage. Accordingly, the term “image” is intended to mean both an image or photograph visible to the human eye and the binary code containing the image information. Temporary storage is to be understood as storage in a memory that is automatically cleared at regular, comparatively short intervals (so-called working memory). Permanent storage is to be understood as storage in a memory that is not intended to be automatically cleared at regular intervals.

The term “automated capturing” is to be understood according to the invention as meaning that a user of the device performs no steps or actions in connection with the capturing, except for placing the block into the holder and removing the block from the holder.

The term “analyze” is to be understood here as the application of an algorithm by the control unit, which can recognize an identification code in the binary code of the captured image.

According to the invention, the camera unit captures an image from the region of the holder and sends it to the control unit. The control unit analyzes the image, and as long as no identification code is recognized when analyzing the image, the camera unit automatically captures again an image from the region of the holder, sends it to the control unit, and the control unit analyzes the image. This sequence is continuously repeated, at least as long as no identification code is recognized. In this case, it does not matter whether a block is located in the holder or not. As soon as a block is placed in the holder, the control unit can recognize the identification code by image analysis. Only when an identification code is recognized does the control unit instruct the camera unit to capture an image of the sample embedded in the block.

The image from the region of the holder comprises at least the region of the holder in which, during proper use of the device according to the invention, the identification code is positioned, so that it is completely captured. However, the image from the region of the holder may also comprise other regions in which neither an identification code or a part thereof nor the embedded sample or a part thereof are positioned during proper use of the device according to the invention, or regions in which, during proper use of the device according to the invention, the embedded sample or a part thereof are positioned.

The image of the embedded sample comprises at least the region of the holder in which, during proper use of the device according to the invention, the embedded sample is positioned, so that it is completely captured. However, the image of the embedded sample may also comprise other regions in which neither an identification code or a part thereof nor the embedded sample or a part thereof are usually positioned, or regions in which, during proper use of the device according to the invention, the identification code or a part thereof are positioned.

The object is completely achieved by the present invention.

The device according to the invention advantageously enables a reduction of the operating steps in the documentation of the section surface of histological blocks and thus a simplification of the workflow. During the preparation of sections by means of a microtome, the user merely has to place the block into the holder. All further steps are carried out automatically by the device. In this way, the user can focus more on producing high-quality sections.

Furthermore, the storage of the images advantageously results in the possibility of accessing them at any later time and, if applicable, from any location. As a result, and through the simplification of the workflow, the time expenditure of the user for the documentation of the blocks and for the subsequent evaluation of the sections is reduced, and the place of work becomes flexible.

In a preferred embodiment, the camera unit comprises at least a first camera and a second camera, wherein the first camera is configured to capture an image of the identification code, and wherein the second camera is configured to capture an image of the sample embedded in the block.

The presence of at least two cameras advantageously makes it possible to adjust the resolution of the image from the region of the holder, which at least captures the region of the holder in which, during proper use of the device according to the invention, the identification code is positioned so that it is completely captured, and the resolution of the image of the embedded sample, in such a way that in each case only the minimum necessary amount of data is generated.

The image of the embedded sample usually requires a higher resolution in order to make the sample, its contours, and, if applicable, internal structures clearly visible even when enlarged. Therefore, the second camera preferably captures the image of the embedded sample with a higher resolution of the captured image than the first camera. In addition, a lower resolution of the image captured by the first camera enables minimization of the amount of data to be sent to and analyzed by the control unit, thereby increasing the working speed.

In a preferred embodiment, the first camera and the second camera are arranged at an angle of about 180° to each other, i.e., they are directed toward each other and capture an image of two substantially opposite sides of the block. Advantageously, this allows the use of commercially available embedding cassettes, which are usually provided with an identification code on the side opposite the section surface (i.e., on the back side).

The term “two substantially opposite sides of a block” is to be understood as two sides of the block whose surfaces are substantially parallel to each other. The two surfaces therefore do not have to be absolutely parallel. For example, the surfaces may be opposite sides of a cuboid or cuboid-like structure. However, the surfaces may also be adjacent sides of a cuboid-like structure, where the surfaces do not extend at a 90° angle to each other but at a smaller angle.

In another preferred embodiment, the control unit is configured to instruct the camera unit to capture the at least one image of the sample embedded in the block only when the recognized identification code is not identical with the identification code recognized immediately before.

The term “identification code recognized immediately before” is to be understood as the identification code that the control unit has recognized during the analysis of that image from the region of the holder which was analyzed by the camera unit immediately prior to the image whose analysis resulted in the recognition of the identification code by the control unit.

This advantageously enables control over the capturing of the image of the embedded sample, since only one image of the embedded sample is captured, and only when a new block has been placed into the holder. In this way, unnecessary repeated capturing of the image of the embedded sample is avoided, which reduces the amount of data to be sent from the camera unit to the control unit, thereby reducing the storage requirements of the control unit and increasing the working speed.

In a further preferred embodiment, the control unit is further configured to discard the at least one image of the sample embedded in the block if the identification code recognized after capturing the at least one image of the sample embedded in the block is not identical with the identification code recognized before capturing the at least one image of the sample embedded in the block, or if, after capturing the at least one image of the sample embedded in the block, no identification code is recognized, or if, after capturing the at least one image of the sample embedded in the block, an artifact or a contamination is recognized in the image.

It may occur that the block is removed or replaced after its identification code has been recognized and before the image of the embedded sample has been captured. In such cases, the image does not show the embedded sample, but either no sample or a sample that is incorrect with respect to the identification code. It may also occur that a contamination or another artifact is present on the image and possibly covers part of the sample. Advantageously, discarding such an image of the embedded sample serves as a quality control. It is thereby avoided that a possibly faulty image is permanently stored. Furthermore, this advantageously reduces the storage requirements of the control unit.

The term “artifact” is to be understood as an object that is visible on a captured image, the visibility of which, however, is not desired, for example because the object covers a part of the sample or of the identification code. An artifact may, for example, be a contamination caused by dust, particles detached from the embedding material, or a human finger.

In a further preferred embodiment, the device is configured to output an acoustic or visual signal when the identification code recognized after capturing at least one image of the sample embedded in the block is identical with the identification code recognized before capturing the at least one image of the sample embedded in the block.

Advantageously, an acoustic or visual signal enables the user to be informed when an image of the embedded sample has been successfully captured and when he or she can replace the respective block with another block.

In a further preferred embodiment, the device further comprises an illuminating unit which is configured to illuminate the region of the holder.

Illumination has the advantage that the image quality is improved. The illuminating unit may comprise diffuse and/or directed light sources.

In a further preferred embodiment, the illuminating unit comprises exclusively diffuse light sources.

Paraffin as embedding material is both light-transmissive and reflective. Excessively strong one-sided illumination, which could cause translucence and/or reflections, can be avoided by designing the light sources as diffuse light sources. An illuminating unit according to this embodiment is, however, also suitable for other embedding materials, such as plastic polymers.

In a preferred embodiment, the illuminating unit comprises a first light source which is provided to illuminate the identification code, or the identification code and the embedded sample, and a second light source which is provided to illuminate the embedded sample.

Separate illumination of the embedded sample by a second light source advantageously enables optimization of the image quality of the image of the embedded sample in comparison with the image from the region of the holder.

A light source may be a bar light. In the present context, a bar light is to be understood as a light source whose light-emitting surface is bar-shaped, i.e., rectangular.

A light source may be a ring light. In the present context, a ring light is to be understood as a ring-shaped light source which can be positioned around the lens of a camera.

A light source may be a light-emitting diode (LED).

In a further preferred embodiment, the shortest distance between two points of the outer boundary of the part of the device which includes at least the holder and the camera unit is less than 40 cm, preferably less than 35 cm, more preferably less than 28 cm.

According to the invention, the device is configured to be as compact as possible. This advantageously enables space-saving placement of the device at the user's workplace. A maximum dimension of the part of the device that includes at least the holder and the camera unit of up to 40 cm has proven to be sufficiently compact.

The control unit and/or the illuminating unit may, but need not, be arranged outside this compact part of the device.

In a further preferred embodiment, the device comprises a mirror system, onto which the camera unit is directed, and which mirrors the region of the holder.

The mirror system comprises at least one mirror.

A mirror system has the advantage that the number of cameras can be reduced and/or the positioning of the cameras relative to the block can be modified. Both of these advantageously enable a compact design of the device.

According to the invention, the object is further achieved by providing a method of automated capturing at least one image of a block which comprises a sample embedded for histological purposes and an identification code, wherein the method comprises the following steps in this order: capturing an image from the region of the holder, analyzing the image from the region of the holder, repeating the preceding steps at least until the identification code is recognized, and capturing at least one image of the sample embedded in the block.

According to the invention, the capturing and analyzing of the image from the region of the holder take place in a loop, a so-called analysis loop. If an identification code is recognized in the image from the region of the holder, a so-called acquisition loop is initiated, during which an image of the sample embedded in the block is captured. The analysis loop can be continuously and automatically repeated after initiation of the acquisition loop, at least however until the identification code has been recognized.

The term “loop” is to be understood as the continuously repeated execution of a defined sequence of method steps.

According to the invention, each loop can be interrupted by a stop command that can be given manually by the user or that is initiated by the control unit after the expiration of a defined period of time.

In a preferred embodiment, the method is carried out by the device according to the invention. In this case, the method as a whole may run in a loop, a so-called method loop. After capturing the image of the embedded sample, further processing or archiving of the one or more images may take place, or one or more additional images may be captured. Processing may include, for example, image processing or image recognition for detecting artifacts on a captured image. As soon as all desired steps have been carried out, the method is executed again, beginning with the analysis loop.

In a further preferred embodiment, the step of capturing at least one image of the sample embedded in the block is carried out only when an identification code is recognized that is not identical with the identification code recognized immediately before.

In this embodiment, the analysis loop is extended by a novelty control. Entry into the acquisition loop occurs only when a new identification code, i.e., one different from the identification code recognized immediately before, has been recognized.

The term “identification code recognized immediately before” is to be understood as the identification code that the control unit recognized in the analysis loop carried out immediately before.

This advantageously enables that only one image of the embedded sample is captured, and only when a new block has been placed in the holder. In this way, unnecessary repeated capturing of the image of the same embedded sample is avoided, which reduces the amount of data to be sent from the camera unit to the control unit, thereby reducing the storage requirements of the control unit and increasing the working speed.

In a further preferred embodiment, the method, after the step of capturing at least one image of the sample embedded in the block, comprises the following steps: capturing an image from the region of the holder, analyzing the image from the region of the holder, discarding the at least one image of the sample embedded in the block, provided that the same identification code is not recognized again or provided that an artifact is recognized.

In this embodiment, the acquisition loop is extended by a quality control. The at least one captured image of the block is not discarded only if, immediately after completion of the capturing of the at least one image of the block, during renewed capturing and analyzing of an image from the region of the holder, an identification code is recognized that is identical to the identification code recognized in the analysis loop carried out immediately before.

If the identification code that is recognized, after the capturing of the image of the embedded sample, in a newly captured image from the region of the holder is not identical with the identification code from the preceding analysis loop, then the image of the embedded sample may not show the embedded sample, but rather an incorrect sample with respect to the identification code. The discarding of such an image of the embedded sample has the advantage that a quality control is carried out.

Discarded images can be reproduced. For example, the user of the device according to the invention can, before completion of his or her work, manually check the images stored by the control unit and, if applicable, find missing images. These can then be specifically reproduced.

As an alternative to a manual check for missing images, the identification code which is recognized as new, the associated image of the embedded sample of which, however, has been discarded, can be stored separately, so that the user can have this block captured again at a later point in time, whereby an active search for missing images is advantageously eliminated for the user.

According to the invention, the object is further achieved by a computer program product comprising program code means which, when executed on a computer, in particular on a control unit, cause the control unit to carry out the method of capturing at least one image of a block which comprises a sample embedded for histological purposes and an identification code.

Further advantages and features become apparent from the following description and the appended drawings. It is understood that the features mentioned above and those yet to be explained below can be used not only in the respective combinations indicated, but also in other combinations without departing from the scope of the present invention.

### BRIEF DESCRIPTION OF THE DRAWINGS

In the following, non-limiting embodiments are explained in detail with reference to the drawings. In the drawings:

FIG. 1 shows a principle representation of a device for the automated capturing of at least one image of a block which comprises a sample embedded for histological purposes and an identification code;

FIG. 2 shows a principle representation of a device for the automated capturing of at least one image of a block which comprises a sample embedded for histological purposes and an identification code, wherein the device comprises a mirror system;

FIG. 3 shows a device for the automated capturing of at least one image of a block which comprises a sample embedded for histological purposes and an identification code, in an exploded view; and

FIG. 4 shows a flow diagram of a method of automated capturing of at least one image of a block which comprises a sample embedded for histological purposes and an identification code.

### DETAILED DESCRIPTION

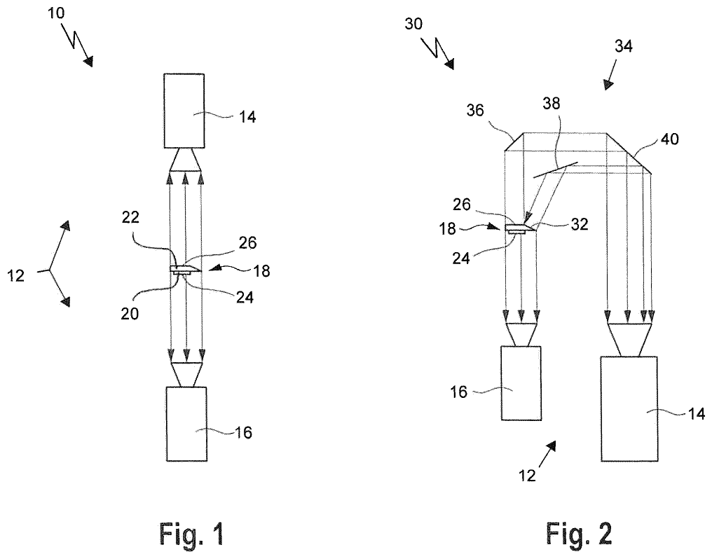

FIG. 1 shows a principle representation of a device 10 for the automated capturing of at least one image of a block which comprises a sample embedded for histological purposes and an identification code. The device 10 comprises a holder (not shown) for a block 18, a camera unit 12, and a control unit (not shown).

The block 18 comprises a sample (not shown) embedded in an embedding material 20. The sample may be any kind of sample the microscopic examination of which is of interest and which is in a cuttable state or has been brought into a cuttable state. Preferably, the sample is tissue of a living being. The embedded sample is visible in a section surface 24 of the block 18. The embedding material 20 may be paraffin or a plastic polymer, preferably paraffin.

Furthermore, the block 18 comprises an embedding cassette 22 which forms the back side 26 of the block 18. The embedding cassette 22 is optional.

An identification code (not shown) is attached to the block 18 by means of which the block 18 can be identified. The identification code may be any kind of optically readable information. The information may, for example, be in the form of a sequence of letters or numbers, a barcode, a QR code, a color code, or a similar format. The identification code may be positioned at any location on the block, with the exception of a location at which the identification code covers-up the embedded sample or a part thereof. Preferably, the identification code is positioned on the back side 26 of the block 18.

The block 18 may have any regular, symmetrical, asymmetrical, or irregular shape. For example, the block 18 may be cuboid, cubic, disk-shaped, or spherical, and is preferably cuboid or cubic. The shape of the block 18, in particular the shape of the back side 26 of the block 18, may be determined by the optional embedding cassette 22. The back side 26 of the block 18 may be flat or may have a relief.

The block 18, for example, has a spatial dimension a×b×c of 42 mm×29 mm×5 mm, wherein the thickness c of the block decreases with the preparation of sections, and a thickness of 5 mm represents the minimum thickness.

The camera unit 12 of the device 10 is configured to repeatedly or continuously capture an image from the region of the holder (not shown), where, during proper use of the device 10, the block 18 is placed. The camera unit 12 comprises a first camera 14 as well as a second camera 16, wherein the first camera 14 is configured to capture an image of the identification code, and wherein the second camera 16 is configured to capture an image of the sample embedded in the block 18. For this purpose, the first camera 14—more precisely its lens or light entrance window—is directed toward the identification code (i.e., toward the back side 26 of the block 18), while the second camera 16—more precisely its lens or light entrance window—is directed toward the embedded sample (i.e., toward the section surface 24 of the block 18).

In the device 10, the cameras 14, 16—more precisely their lenses or light entrance windows—are arranged at an angle of about 180° to each other and are directed toward one another.

In other preferred embodiments, however, the cameras 14, 16 may be arranged at any angle that allows both cameras to capture a sufficiently resolved image of the identification code or, respectively, of the embedded sample.

FIG. 2 shows a principle representation of a device 30 for capturing at least one image of a block 18 which comprises a sample embedded for histological purposes and an identification code, wherein the device 30, in contrast to the device 10, comprises a mirror system 34.

The mirror system 34 comprises at least one mirror. The mirror system 34 of the device 30 comprises three mirrors 36, 38, and 40 and mirrors the back side 26 of the block 18 into the lens or light entrance window of the first camera 14. The back side 26 of the block 18 comprises an inclined surface 32 with respect to the remaining back side 26 of the block 18. This inclined surface is formed by a side surface of the substantially cuboid block 18, the side surface being arranged at an angle of more than 90° to the back side 26 of the block 18 and at an angle of less than 90° to the section surface 24 of the block 18, wherein the side surface under these conditions is referred to in this document as the inclined surface 32 of the back side 26 of the block 18.

The inclined surface 32 of the back side 26 of the block 18 is mirrored by the mirror 38, and the remaining back side 26 of the block 18 is mirrored by the mirror 36 onto the mirror 40 onto which the first camera 14, more precisely its lens or light entrance window, is directed.

The first camera 14 and the second camera 16 are positioned next to each other and aligned parallel. The second camera 16 is directed toward the section surface 24 of the block 18, and the first camera 14 is directed toward the mirror system 34, more precisely toward the mirror 40.

In other preferred embodiments, the camera unit 12 of the device 30 may comprise a single camera which is directed both toward the section surface 24 of the block 18 and toward the mirror system 34.

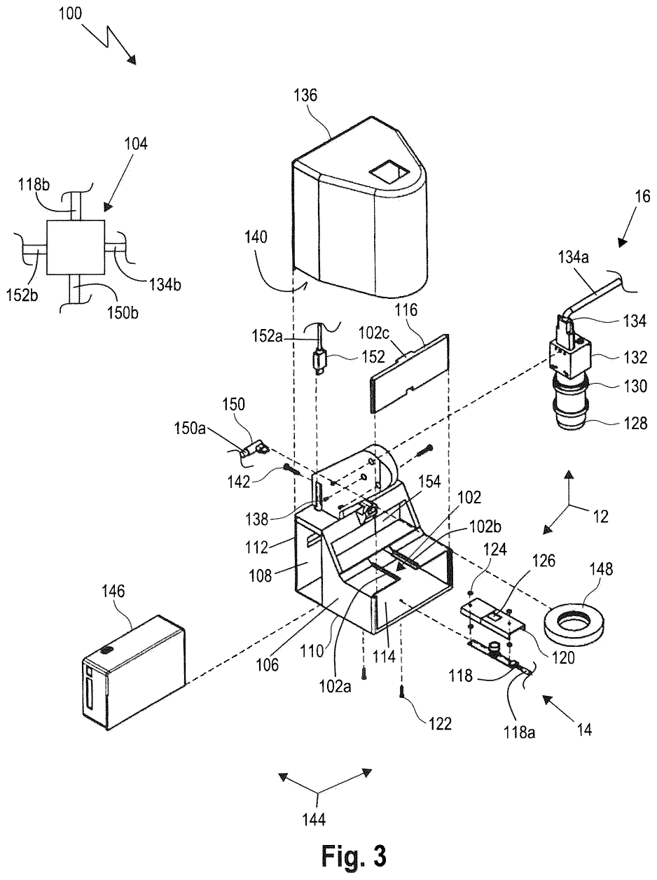

In FIG. 3, a device 100 for capturing at least one image of a block 18 which comprises a sample embedded for histological purposes and an identification code is shown.

The device 100 comprises a holder 102, a camera unit 12—wherein the camera unit 12 comprises a first camera 14 and a second camera 16—and a control unit 104.

The device 100 has the same basic structure as the device 10 of FIG. 1.

The device 100 further comprises a housing 106. The housing 106 is substantially L-shaped and hollow, such that it comprises a lumen 108, a lower horizontal leg 110, and an upper vertical leg 112. The lower, horizontal leg 110 of the L-shaped housing 106 has a substantially flat underside serving as a standing surface and encloses within its lumen 108 the first camera 14. The lower leg 110 of the L-shaped housing 106 has a mounting opening 114 at its outer tip, which extends into the central region of the upper side of the lower leg 110 of the L-shaped housing 106 and is partially closed by a closing piece 116. The closing piece 116 is attached to the housing 106, for example by a plug-in mechanism. The part of the mounting opening 114 that remains when the closing piece 116 is inserted is located in the central region of the upper side of the lower leg 110 of the L-shaped housing 106 and exposes the connecting axis between the first camera 14 and the second camera 16. The edges 102a, 102b, and 102c of the part of the mounting opening 114 that remains after insertion of the closing piece 116 form the holder 102.

In the holder 102, during proper use of the device 100, a block 18 is placed, wherein the holder 102 serves as a support for the block 18. The rectangular surface of the mounting opening 114 is designed to be at least large enough that the embedded sample can be completely captured by the first camera, and at most large enough that, after insertion of the closing piece 116, a block 18 can rest simultaneously on at least two opposite edges of the mounting opening 114.

In another embodiment, the holder 102 is designed to be circular, wherein the circular mounting opening 114 is closed by a transparent material (not shown). In this case, the edges 102a, 102b, 102c of the holder 102 terminate flush, thereby forming a rotational symmetry that is preferably circular or approximately circular. Between the edges 102a, 102b, and 102c of the holder 102, a recess may be formed, the bottom of which is formed by the transparent material. Alternatively, the transparent material may form, toward the block 18, a plane with the L-shaped housing 106. The transparent material may, for example, be glass or a transparent plastic.

In this embodiment, the block 18 can advantageously be placed more easily into the holder 102, since the user does not have to consider its orientation in the horizontal plane.

In other preferred embodiments, the holder 102 may also serve as a mounting in which the block 18 is clamped or snapped into place, such that the camera unit can capture an image of it.

The camera unit 12 of the device 100 comprises the first camera 14 and the second camera 16.

The first camera 14 of the device 100 is, for example, a 5.04-megapixel camera which comprises a lens (not shown), a resolution of 2592 px×1944 px, a minimum object distance of 300 mm, an F/2.8 aperture (not shown), a light sensor (not shown), a field angle of 80°, and a size of 60 mm×9 mm×13.6 mm. The minimum object distance of the first camera 14 can be adjusted or adapted arbitrarily by varying the distance between the lens and the light sensor. A working distance, i.e., a distance between the back side 26 of the block 18 and the lens (not shown) of the first camera 14, is 27.1 mm. The aforementioned characteristics of the first camera 14 have proven to be suitable for resolving an identification code consisting of elements of about 0.34 mm in size.

In other preferred embodiments, the first camera 14 may comprise one or more spacer rings for adjusting or adapting the minimum object distance. Furthermore, the camera specifications may be adapted to the type of the block 18 and to the type of the identification code, such that sufficient resolution is enabled.

In the device 100, the first camera 14 is connected to the control unit 104 via a data interface 118 by means of a cable 118a, 118b and is protected from dust by a first camera cover 120. The data interface 118 and the cable 118a, 118b enable communication and data exchange between the control unit 104 and the first camera 14 and ensure the power supply of the first camera 14. The first camera 14 and the first camera cover 120 are mounted within the lumen 108 and on the bottom of the housing 106 by fastening means, for example screws 122 and nuts 124. The first camera cover 120 comprises an opening 126 and encloses the first camera 14, wherein the connecting axis between the first camera 14 and the second camera 16 remains unobstructed through the opening 126.

The second camera 16 of the device 100 is, for example, a 12-megapixel camera comprising a lens 128 with a focal length of 8 mm and a 1/1.7″ sensor 132 with 4000 px×3036 px and a pixel size of 1.85 μm×1.85 μm. The 1/1.7″ sensor 132 is a light sensor. Furthermore, the second camera 16 comprises two spacer rings 130 with a total thickness of 1 mm. The minimum object distance between the lens 128 and the section surface 24 of the block 18 is about 33.4 mm. A working distance, i.e., a distance between the section surface 24 of the block 18 and the lens 128 of the second camera 16, of 35.4 mm advantageously provides sufficient space to ensure, on the one hand, unobstructed placement and accessibility of the block 18 in the holder 102, and, on the other hand, to keep the spatial dimensions of the device 100 as small or compact as possible. In addition, the camera-specific minimum and maximum object distances are also to be taken into account. Furthermore, the second camera 16 comprises an aperture (not shown), preferably an F/8, F/12, or F/16 aperture. Each of these preferred apertures has proven suitable for ensuring sufficient depth of field for blocks with a thickness c of about 7.87 mm to about 14.81 mm.

In other preferred embodiments, the camera specifications may be adapted to the type of the block 18 and to the type of the identification code, such that sufficient resolution is enabled.

In the device 100, the second camera 16 is connected to the control unit 104 via a data interface 134 by means of a cable 134a, 134b and is protected by a second camera cover 136. The data interface 134 and the cable 134a, 134b enable communication and data exchange between the control unit 104 and the second camera 16 and ensure the power supply of the second camera 16. The second camera 16 is fastened to the housing 106 by fastening means, for example by screws 138. The second camera cover 136 is hollow, has on its underside facing the holder 102 an opening 140, is fastened to the housing 106 by fastening means such as screws 142, and encloses the second camera 16, wherein the connecting axis between the first camera 14 and the second camera 16 remains unobstructed through the opening 140.

In other preferred embodiments, the camera unit 12 may comprise one single camera.

In other preferred embodiments, a transparent material (not shown) may be located between the camera unit 12 or a part of the camera unit 12, in particular the lens 128 of the camera unit 12, or the first camera 14 and/or the second camera 16, in particular the lens of the first camera 14 and/or the lens 128 of the second camera 16, and the holder 102. The transparent material is positioned and dimensioned such that contamination of the lens 128 or of the lenses is prevented or reduced. The transparent material may form part of the holder 102. Contamination may occur, for example, due to pieces detaching from the embedding material, dust, or similar substances. The transparent material may, for example, be glass or a transparent plastic.

Furthermore, the device 100 is equipped with an illuminating unit 144 which is configured to illuminate the region of the holder 102. The illuminating unit 144 comprises a bar light 146 and a ring light 148. The ring light 148 is connected via a plug 150 to the control unit 104 by means of a cable 150a, 150b. The bar light 146 is connected via a plug 152 to the control unit 104 by means of a cable 152a, 152b. Through the cables 150a, 150b and 152a, 152b and the plugs 150 and 152, the illuminating unit 144 is supplied with electrical power.

In other embodiments, the illuminating unit 144 may comprise an LED (not shown), which is configured to illuminate the identification code.

The L-shaped housing 106 has a bar-shaped or rectangular bar-light opening 154 on the front of its upper, vertical leg 112, and the hollow upper leg 112 of the L-shaped housing is filled over its entire length, that is, from the base surface to the upper tip, with the bar light 146. In this way, the bar light 146 illuminates both the section surface 24 of the block 18 facing the second camera 16 and the back side 26 of the block 18 facing the first camera 14 and the lumen 108 of the lower leg 110 of the L-shaped housing 106.

At the tip of the upper leg 112 of the L-shaped housing 106, the ring light 148 is fastened. Screws (not shown) may serve as fastening means. In this way, the ring light 148 illuminates the section surface 24 of the block 18 and thus the sample embedded in the block 18.

In other preferred embodiments, the bar light 146 and the ring light 148 may be connected to the control unit 104 by means of a single, branching cable. The illuminating unit 144 as a whole may also be connected, instead of to the control unit 104, to an external power source, wherein the illuminating unit 144 may be manually switched-on at the device 100, preferably by actuating a button or switch.

A part of the device 100 which does not include the control unit 104 and the cables 118a, 118b, 134a, 134b, 150a, 150b, 152a, 152b has spatial dimensions of 12.5 cm×11.4 cm×21.6 cm. Accordingly, the shortest distance between two points of the outer boundary of the part of the device 100 that includes at least the holder 102 and the camera unit 12 is less than 28 cm.

In other preferred embodiments, the shortest distance between two points of the outer boundary of the part of the device 100 that includes at least the holder 102 and the camera unit 12 may be less than 40 cm, preferably less than 35 cm, more preferably less than 28 cm.

In further preferred embodiments, the entire device 100 or a part of the device 100 which does not include the control unit 104 as well as the cables 118a, 118b, 134a, 134b, 150a, 150b, 152a, 152b and the illuminating unit 144 may have the above-mentioned spatial dimensions of less than 40 cm, preferably less than 35 cm, more preferably less than 28 cm.

The control unit 104 of the device 100 is a computer.

In other preferred embodiments, the control unit 104 may be any type of technical device that comprises at least one processor (CPU) and a temporary memory, for example a so-called working memory, and may further comprise a permanent memory. The control unit may be of decentralized configuration.

In other preferred embodiments, the control unit 104 comprises two secondary control units, each having its own CPU. A first secondary control unit (not shown) is, for example, a computer, and a second secondary control unit (not shown) is installed in a scanner (not shown). The first camera 14, which is part of the camera unit 12, is also installed in the scanner. The term “scanner” thus refers to components that belong both to the camera unit and to other components that belong to the control unit. Only the secondary control unit installed in the scanner gives instructions to the first camera 14.

In other preferred embodiments, the control unit 104 may comprise more than two secondary control units.

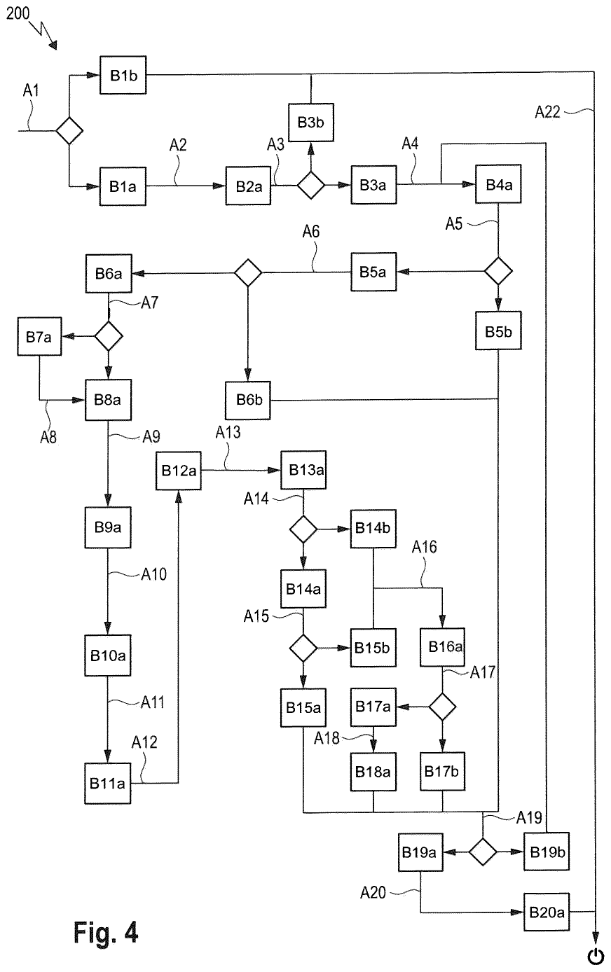

The device 100 performs a method 200, shown in FIG. 4, of the automated capturing of at least one image of a block 18 which comprises a sample embedded for histological purposes and an identification code. Unless otherwise specified, the control unit 104 gives all instructions (reference numerals preceded by “A”) and checks all conditions (reference numerals preceded by “B”) according to instructions from program code means executed on the control unit 104. The program code means may be comprised in a computer program product.

The illuminating unit 144 of the device 100 is switched on when the execution of the program code means is started and is switched off as soon as an instruction (at A22) to terminate the execution of the program code means is given.

The execution of the program code means can be started by the user. When the execution of the program code means is started, the control unit 104 attempts to connect to the camera unit 12, in the device 100 specifically to the second camera 16 (at A1). If this is not successful (at B1b), an instruction to terminate the execution of the program code means is given (at A22). If the second camera 16 is successfully connected to the control unit 104 (at B1a), an instruction is given to the second camera 16 to set parameters, such as exposure time (at A2). The parameters may also be set manually by the user before an instruction to set parameters, such as exposure time, is given to the second camera 16 (at A2). If the parameters have been set (at B2a), the control unit 104 connects to the first camera 14 (at A3). If this is not successful (at B3b), an instruction to terminate the execution of the program code means is given (at A22).

If the first camera 14 has been successfully connected to the control unit 104 (at B3a), the analysis loop begins, by giving the first camera 14 an instruction to capture an image from the region of the holder 102 and to send the image to the control unit 104 (at A4). If the image has been captured and sent to the control unit 104 (at B4a), it is analyzed by the control unit 104 (at A5).

In other preferred embodiments, in which the camera unit 12 comprises only one single camera, the instruction to connect the control unit 104 to the first camera 14 (at A3) as well as the condition that the first camera 14 must be connected to the control unit 104 (at B3a) are omitted. After the successful setting of the parameters of the camera unit 12 (at B2a), the analysis loop begins directly (at A4).

In the method 200 executed by the device 100, if no identification code is recognized (at B5b), it is checked whether a stop command has been given (at A19). Then, the analysis loop is either started again if no stop command has been given (at B19b), or the termination of the execution of the program code means is initiated.

If the control unit 104, during the analysis (at A5), has recognized an identification code (at B5a), it is checked whether the identification code is identical with the identification code that was recognized in the preceding analysis loop (at A6). If an identification code identical to that of the preceding analysis loop is recognized (at B6b), it is checked whether a stop command has been given (at A19). Then, the analysis loop is either started again if no stop command has been given (at B19b), or the termination of the execution of the program code means is initiated.

In other preferred embodiments, for initiating the acquisition loop (at A7), both the instruction to check whether the identification code is identical with the identification code recognized in the preceding analysis loop (at A6) and the condition that an identification code not identical to that of the preceding analysis loop has been recognized (at B6a) may be omitted. The latter check serves as a mechanism to avoid unnecessary repeated storage of identical images from the region of the holder 102.

In the method 200 executed by the device 100, if an identification code not identical to that of the preceding analysis loop is recognized (at B6a), the analysis loop transitions into an acquisition loop. In the acquisition loop, by means of the camera unit 12—in the device 100 specifically by the second camera 16—at least one image of the embedded sample is captured and stored at a permanent storage location assigned to the recognized identification code of the respective block 18, i.e., in a so-called folder. For this purpose, it is checked whether the folder assigned to the identification code exists (at A7). If the folder does not exist (at B7a), an instruction is given to generate the folder (at A8).

If the folder exists or has been generated (at B8a), an instruction is given to the camera unit 12—in the device 100 specifically to the second camera 16—to capture an image of the embedded sample (at A9). If the image of the embedded sample has been captured (at B9a), an instruction is given to store the image of the embedded sample in the folder (at A10). If the image of the embedded sample has been stored in the folder (at B10a), an instruction is given to the camera unit 12—in the device 100 specifically to the first camera 14—to capture an image from the region of the holder 102 (at A11). If the image from the region of the holder 102 has been captured (at B11a), an instruction is given to store the image from the region of the holder 102 in the folder (at A12).

If the image of the embedded sample and the image from the region of the holder 102 have been stored in the folder (at B12a), quality control begins, by giving the camera unit 12—in the device 100 specifically the first camera 14—an instruction to capture an image from the region of the holder 102 and to send it to the control unit 104 (at A13). If the image from the region of the holder 102 has been captured and sent to the control unit 104 (at B13a), this image is analyzed by the control unit 104 (at A14).

If an identification code is recognized (at B14a), it is checked whether this identification code is identical with the identification code recognized in the analysis loop (at A15). If this is the case (at B15a), it is checked whether a stop command has been given (at A19). Then, the analysis loop is either started again if no stop command has been given (at B19b), or the termination of the execution of the program code means is initiated.

If no identification code is recognized (at B14b), or if an identification code is recognized that is not identical with the identification code recognized in the analysis loop (at B15b), or if an artifact is recognized (not shown), an instruction is given to discard the image from the region of the holder and the image of the embedded sample (at A16). If the images have been discarded (at B16a), it is checked whether the folder is empty (at A17). If the folder is empty (at B17a), an instruction is given to discard the folder (at A18). If the folder is not empty (at B17b) or if the folder has been discarded (at B18a), it is checked whether a stop command has been given (at A19). Then, the analysis loop is either started again if no stop command has been given (at B19b), or the termination of the execution of the program code means is initiated.

In other preferred embodiments, after the quality control, i.e., if an identification code has been recognized that is identical with the identification code recognized in the analysis loop (at B15a), the user may be notified by a signal of the successful documentation of the respective block (18), that is, of the non-discarding of the already permanently stored images of the block 18. The signal may, for example, be an acoustic or visual signal.

In further preferred embodiments, the quality control may be omitted, in that, if the image of the embedded sample has been stored in the folder (at B12a), an instruction is given to check whether a stop command has been given (at A19). Then, the analysis loop is either started again if no stop command has been given (at B19b), or the termination of the execution of the program code means is initiated.

In these embodiments, the user can already be notified—if the image of the embedded sample and the image from the region of the holder 102 have been stored in the folder (at B12a)—by a signal of the successful documentation of the respective block 18, that is, of the non-discarding of the already stored images of the block 18. The signal may, for example, be an acoustic or visual signal.

In the method 200 executed by the device 100, a stop command can be manually issued by a user at any time during the execution of the program code means. If a stop command has been issued (at B19a), an instruction is given to disconnect the control unit 104 from the second camera 16 (at A20). If the second camera 16 has been disconnected (at B20a), an instruction is given to terminate the execution of the program code means (at A22). If no stop command has been issued (at B19b), the analysis loop is started again by giving an instruction to the camera unit 12—in the device 100 specifically to the first camera 14—to capture the image from the region of the holder 102 (at A4).

In other preferred embodiments, a stop command may be automatically issued by the control unit 104 if a condition is met, such as the expiration of a defined operating time during which no identification code has been recognized.

What is claimed is:

1. A device for automated capturing of at least one image of a block, the block comprising a sample embedded for histological purposes and an identification code, wherein the device comprises:
a holder for the block,
a camera unit comprising at least one camera,
a control unit,
wherein the camera unit is configured to repeatedly capture an image from the region of the holder and to send it to the control unit,
wherein the control unit is configured to receive and analyze each image,
wherein the control unit, provided that the identification code has been recognized, instructs the camera unit to capture at least one image of the sample embedded in the block.

2. The device according to claim 1, wherein the camera unit comprises at least a first camera and a second camera, wherein the first camera is configured to capture an image of the identification code, and wherein the second camera is configured to capture an image of the sample embedded in the block.

3. The device according to claim 1, wherein the control unit is configured to instruct the camera unit to capture the at least one image of the sample embedded in the block only when the recognized identification code is not identical with the identification code recognized immediately before.

4. The device according to claim 1, wherein the control unit is further configured to discard the at least one image of the sample embedded in the block, if the identification code recognized after capturing the at least one image of the sample embedded in the block is not identical with the identification code recognized before capturing the at least one image of the sample embedded in the block.

5. The device according to claim 1, wherein the control unit is further configured to discard the at least one image of the sample embedded in the block, if no identification code is recognized after capturing the at least one image of the sample embedded in the block.

6. The device according to claim 1, wherein the control unit is further configured to discard the at least one image of the sample embedded in the block, if, after capturing the at least one image of the sample embedded in the block, an artifact is recognized in the image.

7. The device according to claim 1, wherein the device is further configured to output an acoustic or visual signal when the identification code recognized after capturing the at least one image of the sample embedded in the block is identical with the identification code recognized before capturing the at least one image of the sample embedded in the block.

8. The device according to claim 1, further comprising an illuminating unit configured to illuminate the region of the holder.

9. The device according to claim 8, wherein the illuminating unit comprises exclusively diffuse light sources.

10. The device according to claim 8, wherein the illuminating unit comprises a first light source which is provided to illuminate at least one of the following: the identification code or the identification code and the embedded sample, and wherein the illuminating unit comprises a second light source provided to illuminate the embedded sample.

11. The device according to claim 1, wherein a shortest distance between two points of an outer boundary of a part of the device which includes at least the holder and the camera unit is less than 40 cm.

12. The device according to claim 1, wherein a shortest distance between two points of an outer boundary of a part of the device which includes at least the holder and the camera unit is less than 35 cm.

13. The device according to claim 1, wherein a shortest distance between two points of an outer boundary of a part of the device which includes at least the holder and the camera unit is less than 28 cm.

14. The device according to claim 1, further comprising a mirror system, wherein the camera unit is directed onto the mirror system, wherein the mirror system is configured to mirror the region of the holder.

15. A method of automated capturing at least one image of a block, the block comprising a sample embedded for histological purposes and an identification code, wherein the method comprises the following steps in this order:
capturing an image from the region of the holder,
analyzing the image from the region of the holder,
repeating the preceding steps at least until the identification code is recognized, and
capturing at least one image of the sample embedded in the block.

16. The method according to claim 15, wherein the step of capturing at least one image of the sample embedded in the block is carried out only when an identification code is recognized which is not identical with the identification code recognized immediately before.

17. The method according to claim 15, wherein the method, after the step of capturing at least one image of the sample embedded in the block, comprises the following steps:
capturing an image from the region of the holder,
analyzing the image from the region of the holder,
discarding the at least one image of the sample embedded in the block, provided that the same identification code is not recognized again.

18. The method according to claim 15, wherein the method, after the step of capturing at least one image of the sample embedded in the block, comprises the following steps:
capturing an image from the region of the holder,
analyzing the image from the region of the holder,
discarding the at least one image of the sample embedded in the block, provided that an artifact is recognized.

19. A non-transitory computer-readable medium storing instructions that, when executed by a control unit, cause the control unit to carry out a method of automated capturing at least one image of a block, the block comprising a sample embedded for histological purposes and an identification code, the method comprising the following steps in this order:
capturing an image from the region of the holder,
analyzing the image from the region of the holder,
repeating the preceding steps at least until the identification code is recognized, and
capturing at least one image of the sample embedded in the block.
